(19) United States
(12) Patent Application Publication
(10) Pub. No.: US 2010/0297748 A1
(43) Pub. Date: Nov. 25, 2010

(54) INTEGRATED FLUIDIC CIRCUITS

(75) Inventors: Mark Davies, Limerick (IE); Tara Dalton, Limerick (IE)

Correspondence Address: BROWN RUDNICK LLP, ONE FINANCIAL CENTER, BOSTON, MA, 02111, US

(73) Assignee: Stokes Bio Limited, Limerick (IE)

(21) Appl. No.: 12/732,877

(22) Filed: Mar. 26, 2010

Related U.S. Application Data
(63) Continuation-in-part of application No. 12/443,303, filed on Dec. 23, 2009.
(60) Provisional application No. 60/847,683, filed on Sep. 28, 2006.

(51) Int. Cl. C12M 1/00

(52) U.S. Cl. 435/289.1

(57) ABSTRACT

The invention generally relates to an integrated fluidic circuit. In certain embodiments, the circuit includes a main chamber having a withdrawal port, a carrier fluid occupying a volume in the chamber, in which the carrier fluid is immiscible with a sample fluid, and a plurality of liquid bridges disposed within the chamber.

### RELATED APPLICATIONS

The present application is a continuation-in-part of U.S. patent application Ser. No. 12/443,303, filed Mar. 27, 2009, which is a U.S. national phase application of international patent application number PCT/IE2007/000088, filed Sep. 27, 2007 and published in English, which claims priority to and the benefit of U.S. patent application Ser. No. 60/847,683 filed Sep. 28, 2006. The contents of each of these applications are incorporated by reference herein in their entirety.

### FIELD OF THE INVENTION

The present invention generally relates to integrated fluidic circuits.

### BACKGROUND

Microfluidics involves micro-scale devices that handle small volumes of fluids. Because microfluidics can accurately and reproducibly control small fluid volumes, in particular volumes less than 1 μl, it provides significant cost-savings. The use of microfluidics technology reduces cycle times, shortens time-to-results, and increase throughput. Furthermore incorporation of microfluidics technology enhances system integration and automation.

An exemplary microfluidic device involves liquid bridge technology. Liquid bridges allow sample droplet formation or mixing utilizing immiscible fluids. In a liquid bridge, a sample droplet at an end of an inlet port enters a chamber that is filled with a carrier fluid. The carrier fluid is immiscible with the sample droplet. The sample droplet expands until it is large enough to span a gap between inlet and outlet ports. Droplet mixing can be accomplished in many ways, for example, by adjusting flow rate or by introducing a second sample droplet to the first sample droplet, forming an unstable funicular bridge that subsequently ruptures from the inlet port. After rupturing from the inlet port, the mixed sample droplet enters the outlet port, surrounded by the carrier fluid from the chamber. At that point in time, the droplet may be analyzed or undergo further manipulation, for example PCR amplification, QPCR, or immunoassay.

A single system may include numerous liquid bridges, and liquid bridges may be arranged in series and parallel, such that multiple sample solutions may be arrayed with multiple assays of interest. The number of liquid bridges within a single system is dependent on the number of assays to be performed. For example, a system designed to mix a first array having four sample wells with a second array having four sample wells would require 16 liquid bridges. A system designed to mix a first array having 96 sample wells with a second array having 96 sample wells would require over 9,200 liquid bridges. A system designed to mix a first array having 384 sample wells with a second array having 384 sample wells would require over 147,000 liquid bridges.

Thus complexity and size of a system will be determined by a number of liquid bridges that need to be included within the system such that in any pair of positions of first and second sample arrays, all combinations of samples from the first array are mixed with samples from the second array. This presents a problem of numbers, in which the number of liquid bridges and associated connections within a single system become difficult to construct. Additionally, placing such a large number of individual liquid bridges within a single system requires a significant amounts of space, thus system size becomes a problem.

There is a need for systems and devices that can integrate numerous liquid bridges within a single system.

### SUMMARY

The present invention generally relates to integrated fluidic circuits. Devices of the invention include a main chamber having a withdrawal port, a carrier fluid occupying a volume in the chamber, in which the carrier fluid is immiscible with a sample fluid, and a plurality of liquid bridges disposed within the chamber. Devices of the invention allow for multiple liquid bridges to be included within a single chamber having immiscible fluid. Thus multiple liquid bridges may draw from a single supply of carrier fluid, thereby reducing a number of connections associated with each liquid bridge. Further, system size is reduced by integrating multiple liquid bridges within a single chamber. Thus devices of the invention reduce system complexity and system size.

Devices of the invention may further include a supply port in the main chamber, for delivery of the carrier fluid to the chamber. Each of the liquid bridges may include at least one inlet in liquid communication with the main chamber for introducing at least one sample fluid into the main chamber, and at least one outlet in liquid communication with the main chamber, wherein the outlet is separated from the inlet such that the sample fluid forms a droplet wrapped in the carrier fluid prior to entering the outlet. In certain embodiments, each liquid bridge may further include an auxiliary chamber having a withdrawal port in liquid communication with the main chamber, the auxiliary chamber housing a distal portion of the inlet and a proximal portion of the outlet.

Devices of the invention may be configured such that the liquid bridge includes an air bubble in either the main chamber or in at least one of the auxiliary chambers. The air bubble allows for systems of the invention to compensate for pressure changes that may occur within the system without disrupting droplet mixing. Devices of the invention may also be configured with channels of different lengths and different inner diameters connected to the inlet and the outlet of the liquid bridges, thus providing different resistances across the bridges and across the system.

Liquid bridges may be designed to have numerous configurations. Design of the liquid bridge depends on the criteria needed for the application to be performed, e.g., droplet formation or droplet mixing. For example, the liquid bridges in devices of the invention may be designed with two inlets. The first inlet delivers a first sample fluid to the bridge, a second inlet delivers a second sample fluid to the bridge, and the outlet receives a droplet including a mixture of the first sample fluid and the second sample fluid wrapped in the carrier fluid. In a particular embodiment, the first inlet and the outlet are co-axial, and the second inlet is substantially perpendicular to the axis. In another embodiment, the second inlet has a smaller cross-sectional area than the first inlet. Liquid bridges may also be configured such that a first inlet delivers the sample fluid to the bridge, and the second inlet delivers the carrier fluid to the bridge.

Liquid bridges may also be designed to include three inlets. A first inlet delivers a first sample fluid to the bridge, a second inlet delivers a second sample fluid to the bridge, a third inlet delivers the carrier fluid to the bridge, and the outlet receives a droplet including a mixture of the first sample fluid and the second sample fluid wrapped in the carrier fluid. The first sample fluid and a second sample fluid mix at the bridge to form mixed droplets wrapped in carrier fluid that are received by the outlet. In certain embodiments, the withdrawal port of the main chamber withdraws the carrier fluid from between the first and second sample fluids before the first and second sample fluids bridge to the outlet of each liquid bridge so that the first and second sample fluids are caused to mix at each bridge.

Sample droplets may include any type of molecule, e.g., nucleic acids (e.g., DNA or RNA), proteins, antibodies, small organic molecules, small inorganic molecules, or synthetic molecules. In particular embodiments, the droplet includes nucleic acids. In certain embodiments, the first sample fluid and the second sample fluid include different chemical species within an aqueous phase. For example, the first sample fluid may include genetic material and the second sample fluid may include PCR reagents.

Liquid bridges produce wrapped droplets, i.e., sample droplets that are wrapped in an immiscible carrier fluid. Determination of the carrier fluid to be used is based on the properties of the channel and of the sample. If the sample is a hydrophilic sample, the fluid used should be a hydrophobic fluid. An exemplary hydrophobic fluid is oil, such as AS5 silicone oil (commercially available from Union Carbide Corporation, Danbury, Conn.). Alternatively, if the sample is a hydrophobic sample, the fluid to used should be a hydrophilic fluid. One of skill in the art will readily be able to determine the type of fluid to be used based on the properties of the sample.

### BRIEF DESCRIPTION OF THE DRAWINGS

FIG. 1 is a cross-sectional diagram of an embodiment of an integrated fluidic circuit containing a lattice of channels arranged as liquid bridges with a common withdrawal port.

FIG. 2 is a cross-sectional diagram of an embodiment of an integrated fluidic circuit containing an array of liquid bridges with a common withdrawal port. In this embodiment, each bridge is further housed within an auxiliary chamber.

### DETAILED DESCRIPTION

The present invention generally relates to integrated fluidic circuits. Integrated refers to a combination of multiple components to form a unit, such as multiple liquid bridge within a single main chamber. In certain embodiments, integrated fluidic circuits of the invention may be made from many different components that are then assembled to form multiple liquid bridges within a single main chamber. In particular embodiments, the circuit is designed from a single block of material that is fabricated to form the main chamber and the liquid bridges. Exemplary materials for forming the integrated circuits of the invention include TEFLON (commercially available from Dupont, Wilmington, Del.), polytetrafluoroethylene (PTFE; commercially available from Dupont, Wilmington, Del.), polymethyl methacrylate (PMMA; commercially available from TexLoc, Fort Worth, Tex.), polyurethane (commercially available from TexLoc, Fort Worth, Tex.), polycarbonate (commercially available from TexLoc, Fort Worth, Tex.), polystyrene (commercially available from TexLoc, Fort Worth, Tex.), polyetheretherketone (PEEK; commercially available from TexLoc, Fort Worth, Tex.), perfluoroalkoxy (PFA; commercially available from TexLoc, Fort Worth, Tex.), or Fluorinated ethylene propylene (FEP; commercially available from TexLoc, Fort Worth, Tex.). In particular embodiments, the material is PTFE.

Circuits of the invention include a main chamber having a withdrawal port, a carrier fluid occupying a volume in the chamber, in which the carrier fluid is immiscible with a sample fluid, and a plurality of liquid bridges disposed within the chamber. The carrier fluid need not fill the entire volume within the chamber. In particular embodiments, the carrier fluid fills the entire volume within the chamber.

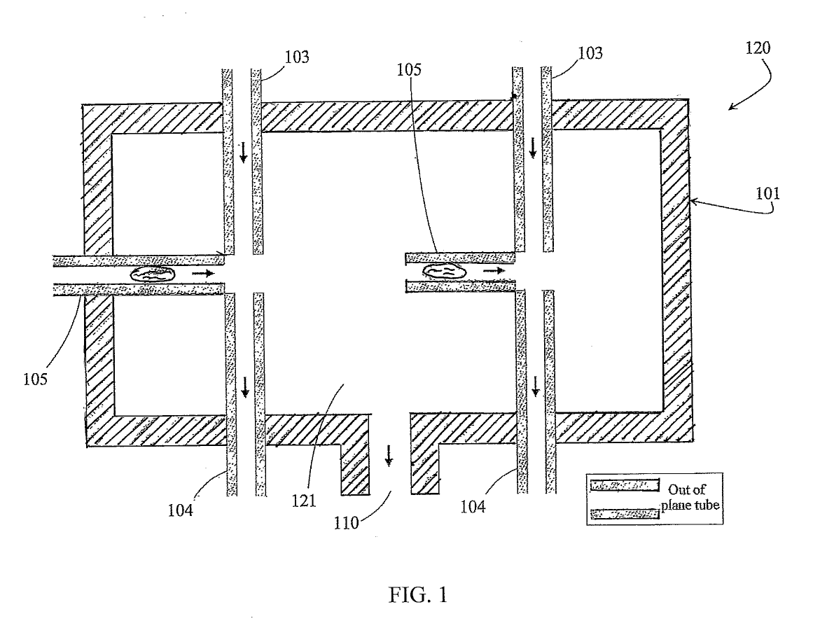

FIG. 1 shows an exemplary embodiment of an integrated fluidic circuit. Main chamber 101 is filled with a carrier fluid that is immiscible with the sample fluid. Determination of the carrier fluid to be used is based on the properties of the channel and of the sample. If the sample is a hydrophilic sample, the fluid used should be a hydrophobic fluid. An exemplary hydrophobic fluid is oil, such as AS5 silicone oil (commercially available from Union Carbide Corporation, Danbury, Conn.). Alternatively, if the sample is a hydrophobic sample, the fluid to used should be a hydrophilic fluid. One of skill in the art will readily be able to determine the type of fluid to be used based on the properties of the sample.

The main chamber 101 includes a common withdrawal port 110 that withdraws the carrier fluid from each of the liquid bridges within the main chamber 101. Thus, instead of connecting outlet 104 to a separate withdrawal system, the liquid bridges are all within main chamber 101 and the withdrawal is to a common withdrawal port 110 from which carrier fluid is withdrawn. Only the carrier fluid is drawn from the port 110 while aqueous phase arriving at inlets 103 and 105 exits to outlet 104. Because of this configuration, the liquid bridges can all be immersed in the carrier fluid and sealed in the main chamber 101, and do not need to be sealed from each other, making manufacture and assembly considerably easier.

In certain embodiments, main chamber 101 includes a supply port that supplies the carrier fluid to the main chamber 101. In alternative embodiments, carrier fluid is supplied to main chamber 101 by individual inlets that are associated with each liquid bridge.

As shown in FIG. 1, within the main chamber 101 is a plurality of liquid bridges. The network of bridges may be repeated many times and may be constructed at very small length scales (e.g., about >10 μm), to form the compact integrated fluidic circuit. The number of liquid bridges within a single circuit is dependent on the number of assays to be performed. For example, a system designed to mix a first array having four sample wells with a second array having four sample wells would require a circuit containing 16 liquid bridges. A system designed to mix a first array having 96 sample wells with a second array having 96 sample wells would require a circuit containing 9,200 liquid bridges, or a series of circuits that when combined would provide for about 9,200 liquid bridges. Thus a single system may include numerous integrated fluidic circuits, and the circuits may be arranged in series and parallel.

In this embodiment only two bridges are shown, for illustration. Each bridge includes two inlets, 103 and 105, and an outlet 104. FIG. 1 shows each liquid bridge configured such that inlet 103 and outlet 104 are co-axial, and inlet 105 is substantially perpendicular to the axis. The bridges may be configured such that inlets 103 and 105 have the same or substantially the same cross-sectional area. Alternatively, the bridges may be configured such that inlet 105 has a smaller cross-sectional area than inlet 103, or that inlet 103 has a smaller cross-sectional area than inlet 105.

In this embodiment, a first sample droplet flows through a first channel to inlet 103 and a second sample droplet flows through a second channel to inlet 105. The first and second droplets arrive at an end of each of inlets 103 and 105 and enter the main chamber that is filled with the carrier fluid. The carrier fluid is immiscible with the sample droplets. The sample droplets expand until large enough to span a gap between inlets 103 and 105 and outlet 104. Droplet mixing occurs as carrier fluid is withdrawn from each bridge by withdrawal port 110, resulting in the first and second sample droplets at inlets 103 and 105 contacting each other, forming an unstable funicular bridge that subsequently ruptures from the inlets 103 and 105. The outlet 110 is configured and positioned so that the withdrawn flow rate is the same for each of the constituent bridges. After rupturing from the inlets 103 and 105, the mixed sample droplet enters the outlet 104, surrounded by the carrier fluid from the chamber, i.e., a wrapped sample droplet. Further description of liquid bridges is shown in Davies et al. (International patent publication number WO 2007/091229), the contents of which are incorporated by reference herein in their entirety.

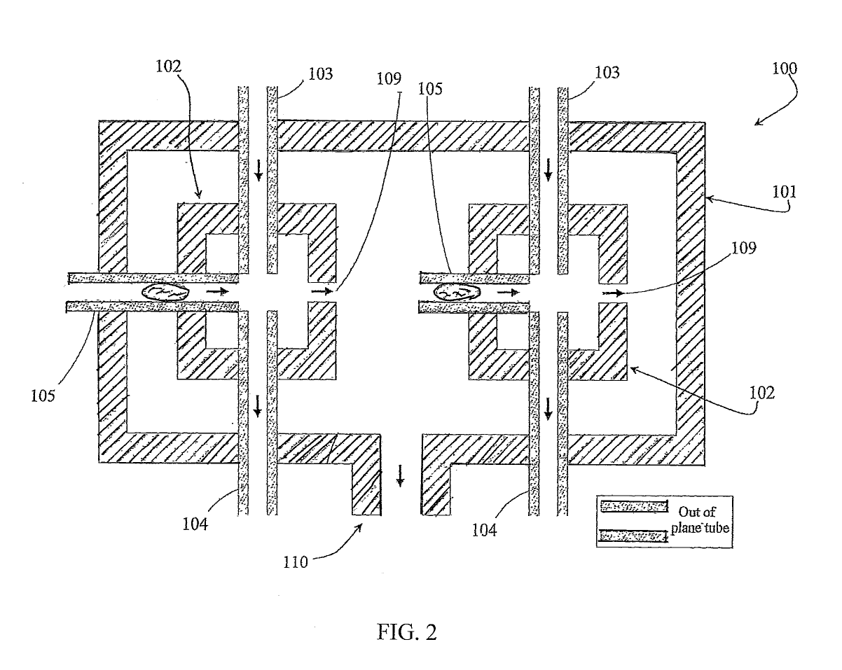

FIG. 2 shows another exemplary embodiment of an integrated fluidic circuit. In this embodiment, the integrated fluidic circuit is constructed such that each liquid bridge includes an auxiliary chamber 102 in fluid communication with a main chamber 101. The auxiliary chamber is configured to house a distal portion of inlets 103 and 105 and a proximal portion of outlet 104. The auxiliary chamber further includes a withdrawal port 109 in liquid communication with the main chamber 101. Instead of connecting outlets 109 to separate withdrawal systems, the withdrawal is to the main chamber 101 from which fluid is withdrawn from the single withdrawal port 110. Only the carrier fluid is drawn from outlets 109 while aqueous phase arriving from inlets 103 and 105 exits to the outlet 104. Because each auxiliary chamber includes its own withdrawal port 109, the auxiliary chamber 102 allows for individual control of extraction of carrier fluid from each liquid bridge.

FIGS. 1 and 2 show exemplary liquid bridges that may be used within integrated fluidic circuits of the invention. However, integrated fluidic circuits of the invention may include liquid bridges having numerous other configurations. Design of the liquid bridge depends on the mixing criteria needed for the application to be performed. For example, the liquid bridges in devices of the invention may be designed with two inlets in which a first inlet delivers sample fluid to the bridge, and a second inlet delivers the carrier fluid to the bridge. In this configuration, the bridges may be used to segment a continuous flow of sample fluid into discrete sample droplets wrapped in the carrier fluid that is immiscible with the sample fluid.

In other embodiments, the liquid bridges may be designed to include three inlets. A first inlet delivers a first sample fluid to the bridge, a second inlet delivers a second sample fluid to the bridge, a third inlet delivers carrier fluid to the bridge, and the outlet receives a droplet including a mixture of the first sample fluid and the second sample fluid wrapped in the carrier fluid. Further configurations for liquid bridges that may be used in the integrated fluidic circuits of the invention are shown in Davies et al. (International patent publication number WO 2007/091229).

Integrated fluidic circuits of the invention may be arranged in series and/or in parallel depending on the criteria needed for the application to be performed.

In certain embodiments, the integrated fluidic circuits of the invention may be configured to include an air bubble. The air bubble allows for circuits of the invention to compensate for pressure changes that may occur within the system without disrupting droplet formation or droplet mixing. In certain embodiments, the air bubble is within the main chamber. In alternative embodiments, at least one of the auxiliary chambers includes an air bubble, or all of the auxiliary chambers includes an air bubble. The air bubble is positioned in the circuit such that it does not interact with any the inlets and/or the outlets within the liquid bridges, i.e., no air is introduced into any of the inlets and/or the outlets.

Circuits of the invention may also be configured such that the inlets and the outlets of the liquid bridges have different lengths and different inner diameters, thereby allowing for different resistances within each bridge, and thus within each circuit. For example, a long narrow inlet provides more resistance to flow than does a short enlarged inlet. Different lengths and inner diameters of inlets and outlets may be configured in each liquid bridge to obtain a desired resistance within a circuit, and thus within a system.

Integrated fluidic circuits may be used in systems having many different components, and the systems may have may include numerous configurations. One of skill in the art will be able to determine required system components based on the application for which the system is being built. An exemplary system, is a system designed for PCR or QPCR. Such a system includes a sample acquisition stage, a thermocycler, and an optical detection device. The integrated fluidic circuit is connected within the system after the acquisition stage and before the thermocycler. The integrated circuit is used to mix sample droplets containing nucleic acids with droplets containing PCR regents in order to form a mixed wrapped droplet that will undergo a PCR reaction at the thermocycler.

A typical PCR or QPCR reaction contains: fluorescent double-stranded binding dye, Taq polymerase, deoxynucleotides of type A, C, G, and T, magnesium chloride, forward and reverse primers and subject cDNA, all suspended within an aqueous buffer. Reactants, however, may be assigned into two broad groups: universal and reaction specific. Universal reactants are those common to every amplification reaction, and include: fluorescent double-stranded binding dye, Taq polymerase, deoxynucleotides A, C, G and T, and magnesium chloride. Reaction specific reactants include the forward and reverse primers and sample nucleic acid.

Sample droplets are formed at the acquisition stage. Any device may be used that results in forming of sample droplets that are wrapped in an immiscible carrier fluid. The wrapped droplets may be formed, for example, by dipping an open ended tube into a vessel. Exemplary sample acquisition devices are shown in McGuire et al. (U.S. patent application Ser. No. 12/468,367). Alternatively, droplets may be formed by liquid bridges. This process involves flowing a continuous plug of sample to a liquid bridge and using the liquid bridge to segment the continuous flow and form the droplets. Thus, such a system may include numerous circuits arranged in series such that a first fluidic circuit is configured to form sample droplets that flow to a second fluidic circuit for mixing.

After droplet mixing, the droplets flow to a thermocycler where the nucleic acids in the droplets are amplified. An exemplary thermocycler and methods of fluidly connecting a thermocycler to a liquid bridge system are shown in Davies et al. (International patent publication numbers WO 2005/023427, WO 2007/091230, and WO 2008/038259, each of which is incorporated by reference herein in its entirety). The thermocycler can be connected to an optical detecting device to detect the products of the PCR reaction. An optical detecting device and methods for connecting the device to the thermocycler are shown in Davies et al. (International patent publication numbers WO 2007/091230 and WO 2008/038259, each of which is incorporated by reference herein in its entirety).

It will be appreciated that the invention provides excellent versatility in bridging of microfluidic flows. The mutual positions of the ports may be changed to optimum positions according to fluidic characteristics and desired outlet flow parameters. For example, there may be adjustment to provide a desired droplet size in outlet flow.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. An integrated fluidic circuit comprising:
a main chamber having a withdrawal port;
a carrier fluid occupying a volume in the chamber, wherein the carrier fluid is immiscible with a sample fluid; and
a plurality of liquid bridges disposed within the chamber.

2. The circuit according to claim 1, wherein the main chamber further comprises a supply port that delivers the carrier fluid to the chamber.

3. The circuit according to claim 1, wherein each liquid bridge comprises:
at least one inlet in liquid communication with the main chamber for introducing at least one sample fluid into the main chamber; and
at least one outlet in liquid communication with the main chamber, wherein the outlet is separated from the inlet such that the sample fluid forms a droplet wrapped in the carrier fluid prior to entering the outlet.

4. The circuit according to claim 3, wherein each liquid bridge further comprises an auxiliary chamber having a withdrawal port in liquid communication with the main chamber, the auxiliary chamber housing a distal portion of the inlet and a proximal portion of the outlet.

5. The circuit according to claim 1, wherein an air bubble is present within the circuit.

6. The circuit according to claim 3, wherein the inlet and the outlet are of different lengths and different inner diameters.

7. The circuit according to claim 1, wherein the carrier fluid is oil.

8. The circuit according to claim 3, wherein the at least one inlet is two inlets.

9. The circuit according to claim 8, wherein a first inlet delivers a first sample fluid to the bridge, a second inlet delivers a second sample fluid to the bridge, and the outlet receives a droplet comprising a mixture of the first sample fluid and the second sample fluid wrapped in the carrier fluid.

10. The circuit according to claim 9, wherein the first inlet and the outlet are co-axial, and the second inlet is substantially perpendicular to the axis.

11. The circuit according to claim 9, wherein the second inlet has a smaller cross-sectional area than the first inlet.

12. The circuit according to claim 9, wherein a first inlet delivers the sample fluid to the bridge, and the second inlet delivers the carrier fluid to the bridge.

13. The circuit according to claim 3, wherein the at least one inlet is three inlets.

14. The circuit according to claim 13, wherein a first inlet delivers a first sample fluid to the bridge, a second inlet delivers a second sample fluid to the bridge, a third inlet delivers the carrier fluid to the bridge, and the outlet receives a droplet comprising a mixture of the first sample fluid and the second sample fluid wrapped in the carrier fluid.

15. The circuit according to claim 14, wherein a first sample fluid and a second sample fluid mix at the bridge to form mixed droplets wrapped in carrier fluid that are received by the outlet.

16. The circuit according to claim 15, wherein the withdrawal port of the main chamber withdraws the carrier fluid from between the first and second sample fluids before the first and second sample fluids bridge to the outlet of each liquid bridge so that the first and second sample fluids are caused to mix at each bridge.

17. The circuit according to claim 9, wherein the first sample fluid and the second sample fluid comprise different chemical species within an aqueous phase.

18. The circuit according to claim 9, wherein the first sample fluid comprises genetic material and the second sample fluid comprises PCR reagents.

19. The circuit according to claim 1, further comprising a flow controller.

20. The circuit according to claim 1, wherein the circuit is fluidly connected to a thermocycler and a detection module for analyzing contents of the droplet.

21. A microfluidic circuit comprising:
a chamber having a withdrawal port;
a carrier fluid occupying a volume in the chamber;
a plurality of inlets in liquid communication with the chamber for introducing at least one sample fluid into the chamber, wherein the sample fluid is immiscible with the carrier fluid; and
a plurality of outlets in liquid communication with the chamber, wherein each outlet has at least one corresponding inlet, and the outlet is separated from the inlet such that the sample fluid forms a droplet wrapped in the carrier fluid prior to entering the outlet.
